(19) United States
(12) Patent Application Publication
(10) Pub. No.: US 2026/0315679 A1
(43) Pub. Date: Oct. 15, 2026

(54) MULTI-FUNCTIONAL LAPAROSCOPIC ASPIRATOR

(71) Applicant: Guangzhou Institute of Cancer Research, the Affiliated Cancer Hospital, Guangzhou Medical University, Guangzhou (CN)

(72) Inventor: Kunpeng LIN, Guangzhou (CN)

(73) Assignee: Guangzhou Institute of Cancer Research, the Affiliated Cancer Hospital, Guangzhou Medical University, Guangzhou (CN)

(21) Appl. No.: 19/464,690

(22) Filed: Jan. 30, 2026

Related U.S. Application Data
(63) Continuation of application No. PCT/CN2025/088037, filed on Apr. 9, 2025.

(30) Foreign Application Priority Data
Mar. 5, 2025 (CN) 2025102544751

(51) Int. Cl. A61M 1/00

(52) U.S. Cl. CPC A61M 1/743; A61M 2205/3327; A61M 2205/3331

(57) ABSTRACT

A multi-functional laparoscopic aspirator includes a holding part and a suction tube fixedly connected thereto, wherein the suction tube communicates with a negative pressure pump through a pipe and with a compression pump through a pipe. The suction tube includes first and second tubes communicating with the negative pressure pump and the compression pump through pipes, respectively. A clamping mechanism for clamping surrounding tissues is disposed at an end of the suction tube away from the holding part. The first tube is located above the second tube. A pressure conversion mechanism for communicating based on a pressure difference between interiors of the first and second tubes is communicated therebetween. A suction port communicates with an end of the first tube away from the negative pressure pump, and a number of nozzles communicate with an end of the second tube away from the compression pump.

### TECHNICAL FIELD

The present invention relates to the field of medical device technology, and specifically relates to a multi-functional laparoscopic aspirator.

### BACKGROUND

In laparoscopic surgery, a laparoscopic aspirator can not only perform irrigation within the pelvic and abdominal cavities and aspirate fluids, residual blood, and smoke within the pelvic and abdominal cavities to maintain the surgical field of view, but also use the aspirator to perform operations such as blocking, pressing, picking, and separating surrounding tissues to assist in the progress of the surgical operation.

However, during the aspiration process of the aspirator, if the suction force is insufficient, fluids, residual blood, or smoke cannot be smoothly aspirated; if the aspiration intensity is too high or due to misoperation, damage may be caused to surrounding tissues. At the same time, impurities such as blood clots are easily inhaled during the aspiration process to block the pipe, affecting work efficiency. Therefore, there are high requirements for the experience of the operator; unskilled operation may lead to the suction of visceral tissues or omental tissues within the abdominal cavity, resulting in blockage of the aspiration head, retention of accumulated blood and fluid, and even causing bleeding of visceral tissues within the laparoscope. Therefore, the present invention provides a multi-functional laparoscopic aspirator, which can automatically balance the negative pressure suction force during the aspiration process, reduce operation difficulty, and maintain pipe patency.

### SUMMARY

To solve the above problems, the present invention provides a multi-functional laparoscopic aspirator for automatically balancing the negative pressure suction force during a suction process, reducing operation difficulty, and maintaining pipe patency.

In order to achieve the above object, the technical solution of the present invention is as follows: a multi-functional laparoscopic aspirator, comprising a holding part and a suction tube, wherein the holding part is fixedly connected to the suction tube, the suction tube communicates with a negative pressure pump for generating negative pressure suction through a pipe, and the suction tube also communicates with a compression pump for pumping irrigation fluid through a pipe. The suction tube includes a first tube and a second tube; the first tube communicates with the negative pressure pump through a pipe, and the second tube communicates with the compression pump through a pipe.

A clamping mechanism for clamping surrounding tissues is disposed at an end of the suction tube away from the holding part.

The first tube is located above the second tube. A pressure conversion mechanism for communicating based on a pressure difference between an interior of the first tube and an interior of the second tube is communicated between the first tube and the second tube. A suction port communicates with an end of the first tube away from the negative pressure pump, and a number of nozzles communicate with an end of the second tube away from the compression pump.

The technical principle of the above solution is as follows: based on the Bernoulli principle, it is known that the first tube aspirates external fluids, residual blood, smoke, and other tissues through the negative pressure suction force generated by the negative pressure pump. The first tube is in a negative pressure state, while the second tube performs irrigation by controlling the opening of the nozzles. In a normal state, the fluid flow rate in the first tube is fast and the pressure is small, while the second tube does not perform irrigation and the flow rate is slow or static and the pressure is large, so as to form a pressure difference. When the first tube is blocked, the communication state is controlled through the pressure conversion mechanism, so as to use the irrigation fluid inside the second tube to perform dredging treatment on the first tube to ensure that the first tube is unobstructed.

Adopting the above solution has the following beneficial effects:

This solution uses the clamping mechanism on the suction tube to clamp surrounding tissues, which facilitates the separation of the clamped tissue from the surrounding tissues to meet the needs of laparoscopic surgery, and at the same time uses the first tube and the second tube to perform aspiration and irrigation around the clamped tissue to facilitate improving the clarity of the surgical field of view.

In this solution, during the use process by the operator, the pressure conversion mechanism is used to dredge the first tube when a blockage occurs, so as to reduce manual cleaning operations and improve the smoothness during the surgical operation process.

This solution uses the pressure condition during the aspiration process to perform automatic control. The operator can focus more on negative pressure suction force control and aspiration, reducing operation difficulty and facilitating control by the operator.

Further, the clamping mechanism includes a first block and a second block, the second block is located above the first block, the first block is fixedly connected to the suction tube, and the second block is rotatably matched with the suction tube.

The nozzles are located at a side of the second block close to the first block, and the suction port is located at an end of the second block away from the suction tube.

A press handle is rotatably matched with an end of the holding part away from the suction tube, an extension block fixedly connected to the holding part is further disposed at one side of the press handle, a balloon is disposed between the extension block and the press handle, and the balloon communicates with a gas pipe. An end of the gas pipe away from the balloon communicates with a pneumatic telescopic rod, the pneumatic telescopic rod is hinged to an end of the second block away from the suction port, and a first torsion spring for resetting is disposed at a rotation position of the second block and the suction tube.

When the press handle squeezes the balloon, the pneumatic telescopic rod extends out and the second block moves away from the first block; when the balloon resets to push the press handle to move, the pneumatic telescopic rod retracts and the second block moves close to the first block.

Beneficial effect: Irrigation fluid is sprayed through the nozzles to wash substances attached to the first block or substances such as residual blood of surrounding tissues. The operator uses the press handle to squeeze the balloon to aspirate around the clamped tissue through the rotating second block, so as to reduce the generation of fluids, residual blood, smoke, and the like around the clamped tissue, facilitating the subsequent abdominal surgery.

Further, the pressure conversion mechanism includes a number of exchange tubes and a closing plate, and the exchange tubes are located between the first tube and the second tube. A locking slot is disposed at a bottom of each exchange tube, a diameter of the locking slot is greater than a diameter of the exchange tube, the closing plate is rotatably matched with the suction tube, and the closing plate is located within the locking slot.

A shaft is fixedly connected to one side of the closing plate, the shaft is rotatably matched with the suction tube, a second torsion spring is sleeved on the shaft, a sleeve is slidably matched within the exchange tube, and a first spring is fixedly connected between two sides of the sleeve and the first tube.

When a negative pressure suction force within the first tube is in a normal state, a top of the sleeve is located within the exchange tube; when the negative pressure suction force within the first tube is greater than the normal state, the top of the sleeve is located within the first tube.

Beneficial effect: In a normal state, since the pressure of the second tube is greater than the pressure of the first tube, the pressure difference between the two pushes the closing plate to close the exchange tube. When a blockage occurs inside the first tube, the closing plate no longer closes the exchange tube at the blockage, allowing irrigation fluid to enter the interior of the first tube to wash both sides to dredge the blockage of the first tube.

At the same time, through the change in the negative pressure difference between the first tube and the second tube, the sleeve is pushed to overcome the resistance of the first spring, so as to reduce the diameter of the first tube or close the first tube through the sleeve, thereby realizing automatic balancing of the damage that may be caused to surrounding tissues by the problem of excessive negative pressure suction force.

Further, the shaft of the closing plate is located at a side of the second tube close to the compression pump.

Beneficial effect: Utilizing the impact force generated when the irrigation fluid inside the second tube flows, the closing plate is closed to form a temporary impact water flow to wash blocked impurities inside the first tube, so as to facilitate achieving the patency of the first tube.

Further, a shaking plate corresponding to the exchange tube is disposed at a top of the first tube, a loosening slot is opened at the top of the first tube, the shaking plate is slidably matched with the loosening slot, and a number of second springs are connected between the shaking plate and the suction tube.

Beneficial effect: When the irrigation fluid inside the second tube enters the interior of the first tube, it contacts the shaking plate to loosen the blockage position near the exchange tube in the first tube; the shaking plate is pushed to shake by the resetted second springs to facilitate maintaining the patency of the first tube.

Further, an auxiliary cavity is further opened within the suction tube, the auxiliary cavity is located between adjacent exchange tubes, and the auxiliary cavity is located above the closing plate close to the shaft, a pushing block is fixedly connected to the shaft, and the pushing block is slidably matched with the auxiliary cavity. A pushing rod is slidably matched within the auxiliary cavity, the pushing rod is located above the shaft, and a third spring is sleeved on the pushing rod.

When the closing plate is perpendicular to the exchange tube, the pushing block is separated from the pushing rod; when the closing plate is parallel to the exchange tube, the pushing block abuts against the pushing rod.

Beneficial effect: Utilizing the rotation of the closing plate, the shaft is used to drive the pushing block to rotate, the pushing block is used to push the pushing rod to move, and the pushing rod is used to penetrate deep into the first tube to loosen the blockage position near the exchange tube in the first tube to ensure the patency of the first tube.

Further, it further includes a control panel located inside the holding part, the control panel is electrically connected to a first pressure sensor and a speaker, the first pressure sensor is located at a side of the closing plate close to the exchange tube, and the speaker is located at one side of the holding part.

The first pressure sensor is configured to measure first pressure data at a top of the closing plate in real time and send the first pressure data to the control panel, the control panel is configured to compare the first pressure data with a set first standard value, if the first pressure data is greater than the first standard value, a standby instruction is issued to the speaker, and if the first pressure data is less than the first standard value, a reminder instruction is sent to the speaker.

Beneficial effect: By comparing the pressure data of the closing plate, it is determined whether a blockage occurs inside the first tube, so as to remind the operator to adjust the surgical process and maintain the surgical field of view.

Further, the control panel is further electrically connected to a second pressure sensor, and the second pressure sensor is located between the two sides of the sleeve and the first spring.

The second pressure sensor is configured to measure second pressure data when the sleeve applies pressure in real time and send the second pressure data to the control panel; the control panel is configured to compare the second pressure data with a set second standard value, if the second pressure data is greater than the second standard value, a warning instruction is sent to the speaker, and if the second pressure data is less than the second standard value, a standby instruction is sent to the speaker.

Beneficial effect: By detecting the condition of the second pressure data when pressure is applied to both sides of the sleeve, it is determined whether the sleeve moves, so as to remind the operator that the negative pressure suction force inside the first tube is too high, and remind the operator to perform active adjustment.

Further, a number of buttons for adjusting working power of the negative pressure pump and the compression pump are disposed on the press handle, and the buttons are located at upper and lower sides of the press handle.

Beneficial effect: Through the buttons on the press handle, while the operator controls the second block to perform clamping through the press handle, it is convenient for the operator's fingers to move up and down, and it is convenient to realize the adjustment of the working power of the negative pressure pump and the compression pump.

Further, the nozzles are located at two sides of a top of the second block.

Beneficial effect: Surrounding tissues are washed through the cleaning fluid sprayed from the nozzles on both sides to reduce impurities attached to the surfaces of the tissues clamped by the first block and the second block, realizing timely cleaning.

Further, a first electromagnetic valve communicates with the suction port, the first electromagnetic valve is electrically connected to the control panel, and a normal state of the first electromagnetic valve is a normally open state.

A cleaning pipe communicates between an end of the first tube close to the suction port and the second tube, a second electromagnetic valve communicates within the cleaning pipe, the second electromagnetic valve is electrically connected to the control panel, and a normal state of the second electromagnetic valve is a normally closed state.

Beneficial effect: The control panel controls the opening and closing of the first electromagnetic valve and the second electromagnetic valve to realize the closing of the first tube and the communication between the first tube and the second tube, so as to use the irrigation fluid of the second tube to wash the impurities remaining inside the first tube to ensure the patency of the first tube.

Further, it further includes a locking mechanism, the locking mechanism includes a locking shaft corresponding to the closing plate, and the locking shaft is rotatably matched with the suction tube.

A moving cavity is opened within the suction tube, the moving cavity is located at one side of the shaft, a first magnet layer is fixedly connected to an end of the locking shaft away from the shaft, the first magnet layer is located within the moving cavity, and an electromagnet is disposed within the moving cavity, the electromagnet being electrically connected to the control panel.

When the electromagnet is not energized, the locking shaft is separated from the closing plate; when the electromagnet is energized, the locking shaft abuts against the closing plate.

Beneficial effect: The locking shaft is used to abut against the closing plate to fix the position of the closing plate so that it does not change, so as to facilitate realizing the washing and cleaning of the first tube.

Further, a number of anti-slip grooves are opened on the extension block.

Beneficial effect: The friction between the operator's hand and the extension block during the holding process is increased through the anti-slip grooves to facilitate the holding stability of the operator and facilitate the operator to drive the suction tube to move.

Additional aspects and advantages of the present invention will be given in part in the following description, and in part will become apparent from the following description or learned through the practice of the present invention.

### BRIEF DESCRIPTION OF THE DRAWINGS

FIG. 1 is an axonometric view of an embodiment of a multi-functional laparoscopic aspirator of the present invention;

FIG. 2 is a top view of an embodiment of a multi-functional laparoscopic aspirator of the present invention;

FIG. 3 is a schematic cross-sectional view in the direction A-A in FIG. 2;

FIG. 4 is an enlarged schematic view of part B in FIG. 3.

The reference numerals in the specification drawings include: 1. holding part; 11. speaker; 12. press handle; 13. pipe; 14. button; 15. balloon; 16. gas pipe; 17. pneumatic telescopic rod; 2. suction tube; 21. first tube; 22. second tube; 23. cleaning pipe; 24. second electromagnetic valve; 25. shaking plate; 26. second spring; 3. first block; 31. second block; 32. nozzle; 33. suction port; 34. first electromagnetic valve; 4. exchange tube; 41. sleeve; 42. first spring; 5. closing plate; 51. shaft; 52. pushing block; 6. pushing rod; 61. third spring; 7. locking shaft.

### DETAILED DESCRIPTION

The technical solution of the present invention will be clearly and completely described below with reference to the accompanying drawings. Obviously, the described embodiments are part of the embodiments of the present invention, but not all of the embodiments. Based on the embodiments in the present invention, all other embodiments obtained by those of ordinary skill in the art without making creative labor belong to the protection scope of the present invention.

In the description of the present invention, it should be noted that the orientations or positional relationships indicated by the terms "center", "upper", "lower", "left", "right", "vertical", "horizontal", "inner", "outer", etc., are based on the orientations or positional relationships shown in the drawings, and are only for the convenience of describing the present invention and simplifying the description, rather than indicating or implying that the indicated device or element must have a specific orientation, be constructed and operated in a specific orientation, and therefore cannot be understood as a limitation on the present invention. In addition, the terms "first", "second", and "third" are used for descriptive purposes only and cannot be understood as indicating or implying relative importance.

In the description of the present invention, it should be noted that unless otherwise clearly specified and limited, the terms "installation", "interconnection", and "connection" should be understood in a broad sense, for example, it may be a fixed connection, a detachable connection, or an integral connection; it may be a mechanical connection or an electrical connection; it may be a direct connection or an indirect connection through an intermediate medium, and it may be internal communication between two elements. For those of ordinary skill in the art, the specific meanings of the above terms in the present invention can be understood in specific situations.

The specific embodiments are described in further detail below:

### Embodiment 1

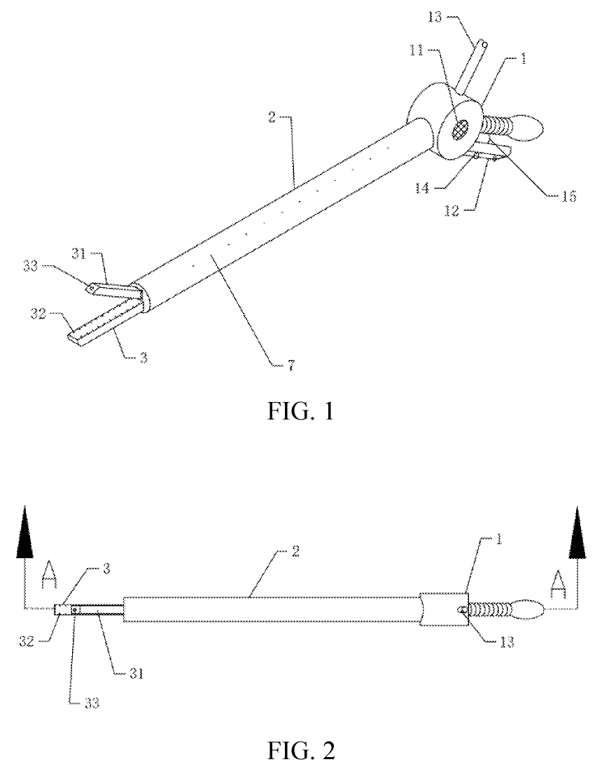

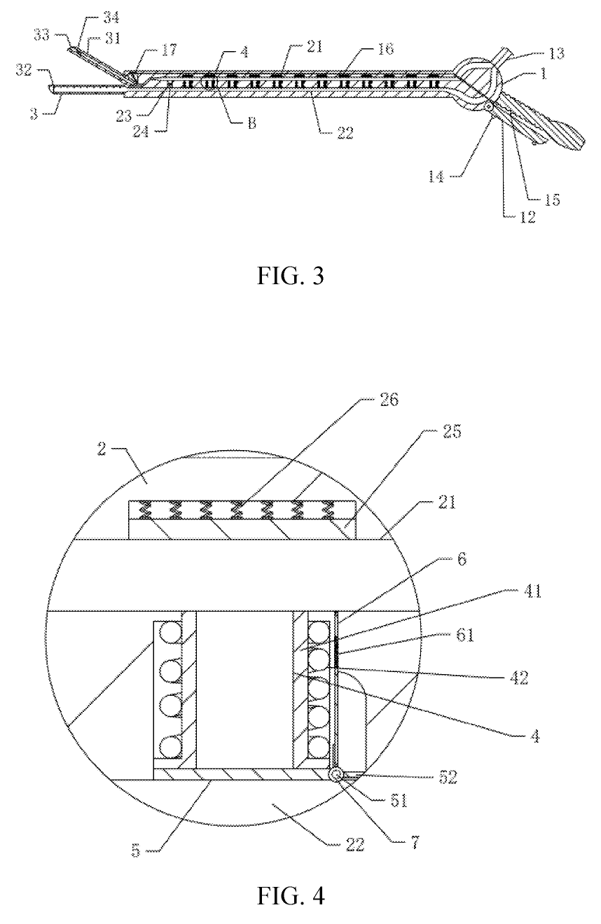

As shown in FIGS. 1 to 4: a multi-functional laparoscopic aspirator, comprising a holding part 1 and a suction tube 2, the holding part 1 is welded with the suction tube 2, the suction tube 2 communicates with a negative pressure pump (not shown in the figure) for generating negative pressure suction through a pipe 13, the suction tube 2 also communicates with a compression pump (not shown in the figure) for pumping irrigation fluid through a pipe 13, and the suction tube 2 includes a first tube 21 and a second tube 22. The first tube 21 communicates with the negative pressure pump through a pipe 13, and the second tube 22 communicates with the compression pump through a pipe 13; the negative pressure pump and the compression pump are prior art and will not be repeated in this embodiment.

A clamping mechanism for clamping surrounding tissues is disposed at an end of the suction tube 2 away from the holding part 1; the first tube 21 is located above the second tube 22, and a pressure conversion mechanism for communicating based on a pressure difference between an interior of the first tube 21 and an interior of the second tube 22 is communicated between the first tube 21 and the second tube 22; and a suction port 33 communicates with an end of the first tube 21 away from the negative pressure pump, and a number of nozzles 32 communicate with an end of the second tube 22 away from the compression pump.

Wherein, the clamping mechanism includes a first block 3 and a second block 31, the second block 31 is located above the first block 3, the first block 3 is integrally formed with the suction tube 2, and the second block 31 is rotatably matched with the suction tube 2; the nozzles 32 are located at a side of the second block 31 close to the first block 3, and the suction port 33 is located at an end of the second block 31 away from the suction tube 2; and the nozzles 32 are located at two sides of a top of the second block 31.

A press handle 12 is rotatably matched with an end of the holding part 1 away from the suction tube 2, and a number of buttons 14 for adjusting working power of the negative pressure pump and the compression pump are disposed on the press handle 12, the buttons 14 being located at upper and lower sides of the press handle 12. An extension block integrally formed with the holding part 1 is further disposed at one side of the press handle 12, and a number of anti-slip grooves are opened on the extension block.

A balloon 15 is disposed between the extension block and the press handle 12, and the balloon 15 communicates with a gas pipe 16; an end of the gas pipe 16 away from the balloon 15 communicates with a pneumatic telescopic rod 17, the pneumatic telescopic rod 17 is hinged to an end of the second block 31 away from the suction port 33, and a first torsion spring for resetting is disposed at a rotation position of the second block 31 and the suction tube 2, one end of the first torsion spring being welded with the second block 31 and the other end of the first torsion spring being welded with the suction tube 2; when the press handle 12 squeezes the balloon 15, the pneumatic telescopic rod 17 extends out and the second block 31 moves away from the first block 3; when the balloon 15 resets to push the press handle 12 to move, the pneumatic telescopic rod 17 retracts and the second block 31 moves close to the first block 3.

The pressure conversion mechanism includes a number of exchange tubes 4 and a closing plate 5, and the exchange tubes 4 are located between the first tube 21 and the second tube 22; a locking slot (not shown in the figure) is disposed at a bottom of each exchange tube 4, a diameter of the locking slot is greater than a diameter of the exchange tube 4, the closing plate 5 is rotatably matched with the suction tube 2, and the closing plate 5 is located within the locking slot; a shaft 51 is welded to one side of the closing plate 5, the shaft 51 is rotatably matched with the suction tube 2, a second torsion spring is sleeved on the shaft 51, a sleeve 41 is slidably matched within the exchange tube 4, and a first spring 42 is fixedly connected between two sides of the sleeve 41 and the first tube 21. Moreover, the shaft 51 of the closing plate 5 is located at a side of the second tube 22 close to the compression pump.

When a negative pressure suction force within the first tube 21 is in a normal state, a top of the sleeve 41 is located within the exchange tube 4; when the negative pressure suction force within the first tube 21 is greater than the normal state, the top of the sleeve 41 is located within the first tube 21.

The specific implementation process is as follows: First, the operator drives the holding part 1 and the suction tube 2 to move by holding the extension block, and uses the anti-slip grooves on the extension block to improve the holding stability of the operator. During use, through the buttons 14 on the press handle 12, it is convenient for the operator's fingers to move up and down, and it is convenient to realize the adjustment of the working power of the negative pressure pump and the compression pump.

During the aspiration process of the suction tube 2, based on the Bernoulli principle, it is known that the first tube 21 aspirates external fluids, residual blood, smoke, and other tissues through the negative pressure suction force generated by the negative pressure pump. The first tube 21 is in a negative pressure state, while the second tube 22 performs irrigation by controlling the opening of the nozzle 32. In a normal state, the fluid flow rate inside the exchange tube 4 is fast and the pressure is small, so that a negative pressure adsorption force is formed at the junction of the exchange tube 4 and the second tube 22, which is convenient for the closing plate 5 to close the exchange tube 4. When the second tube 22 does not perform irrigation, the flow rate inside the second tube 22 is slow or static and the pressure is large, so as to form a pressure difference. At this time, since the pressure of the second tube 22 is greater than the pressure of the first tube 21, the pressure difference between the two pushes the closing plate 5 to close the exchange tube 4.

When a blockage occurs inside the first tube 21, the first tube 21 between the blockage and the suction port 33 communicates with the outside, reducing the pressure difference between the interior of the first tube 21 and the interior of the second tube 22, and utilizing the torsional force of the torsion spring, the closing plate 5 no longer closes the exchange tube 4 at the blockage, making the high pressure inside the second tube 22 (compared with the interior of the first tube 21) allow irrigation fluid to enter the interior of the first tube 21 to wash both sides to dredge the blockage of the first tube 21.

At the same time, through the change in the negative pressure difference between the first tube 21 and the second tube 22, the sleeve 41 is pushed to overcome the resistance of the first spring 42. The greater the pressure difference between the two, the greater the moving distance of the closing plate 5, so as to reduce the diameter of the first tube 21 or close the first tube 21 through the sleeve 41, thereby realizing automatic balancing of the damage that may be caused to surrounding tissues by the problem of excessive negative pressure suction force and reducing operation difficulty.

When the second tube 22 communicates with the first tube 21 through the exchange tube 4, utilizing the impact force generated when the irrigation fluid inside the second tube 22 flows, when the closing plate 5 is opened due to the decrease in air pressure difference, the irrigation fluid inside the second tube 22 is used to push the closing plate 5 to close, so as to form a temporary impact water flow to wash blocked impurities inside the first tube 21, so as to facilitate achieving the patency of the first tube 21.

When tissue separation is performed through the clamping mechanism, the operator uses the press handle 12 to squeeze the balloon 15 to control the clamping degree between the second block 31 and the first block 3, so as to use the first block 3 and the second block 31 to clamp surrounding tissues, which is convenient for the separation of clamped tissue from the surrounding tissues. At the same time, aspiration is performed around the clamped tissue through the rotating second block 31 to reduce the generation of fluids, residual blood, smoke, and the like around the clamped tissue, facilitating the subsequent abdominal surgery. Then, irrigation fluid is sprayed through the nozzle 32 to wash substances attached to the first block 3 or substances such as residual blood of surrounding tissues to ensure the surgical field of view.

### Embodiment 2

The difference from Embodiment 1 is that a shaking plate 25 corresponding to the exchange tube 4 is disposed at a top of the first tube 21, a loosening slot is opened at the top of the first tube 21, the shaking plate 25 is slidably matched with the loosening slot, and a number of second springs 26 are connected between the shaking plate 25 and the suction tube 2.

The specific implementation process is as follows: When the irrigation fluid inside the second tube 22 enters the interior of the first tube 21, it first contacts the shaking plate 25 and overcomes the resistance of the second spring 26 to make the diameter of the first tube 21 expand, so as to loosen the blockage position near the exchange tube 4 in the first tube 21; after the fluid in the second tube 22 is discharged, the resetted second springs 26 push the shaking plate 25 to shake, making the blockage position near the exchange tube 4 in the first tube 21 loosen, so as to facilitate maintaining the patency of the first tube 21.

### Embodiment 3

The difference from Embodiment 2 is that an auxiliary cavity (not marked in the figure) is further opened within the suction tube 2, the auxiliary cavity is located between adjacent exchange tubes 4, and the auxiliary cavity is located above the closing plate 5 close to the shaft 51, a pushing block 52 is welded to the shaft 51, and the pushing block 52 is slidably matched with the auxiliary cavity; a pushing rod 6 is slidably matched within the auxiliary cavity, the pushing rod 6 is located above the shaft 51, and a third spring 61 is sleeved on the pushing rod 6, one end of the third spring 61 being welded with the pushing rod 6 and the other end of the third spring 61 being welded with the suction tube 2; when the closing plate 5 is perpendicular to the exchange tube 4, the pushing block 52 is separated from the pushing rod 6; when the closing plate 5 is parallel to the exchange tube 4, the pushing block 52 abuts against the pushing rod 6.

The specific implementation process is as follows: During the process of pushing the closing plate 5 to rotate using the pressure difference between the first tube 21 and the second tube 22, utilizing the rotation of the closing plate 5, the shaft 51 is used to drive the pushing block 52 to rotate, the pushing block 52 is used to push the pushing rod 6 to move, and the pushing rod 6 is used to penetrate deep into the first tube 21 to loosen the blockage position near the exchange tube 4 in the first tube 21 to ensure the patency of the first tube 21.

### Embodiment 4

The difference from Embodiment 3 is that it further includes a control panel (not shown in the figure) located inside the holding part 1, the control panel is electrically connected to a first pressure sensor (not shown in the figure) and a speaker 11, the first pressure sensor is located at a side of the closing plate 5 close to the exchange tube 4, and the speaker 11 is located at one side of the holding part 1; in another embodiment, the speaker 11 is an LED light, and different colors or frequencies of light are emitted through the LED light to remind the operator to perform corresponding operations to ensure surgical safety.

The first pressure sensor is configured to measure first pressure data at a top of the closing plate 5 in real time and send the first pressure data to the control panel, the control panel is configured to compare the first pressure data with a set first standard value, if the first pressure data is greater than the first standard value, a standby instruction is issued to the speaker 11, and if the first pressure data is less than the first standard value, a reminder instruction is sent to the speaker 11.

For example, the detection is performed based on the contact between the closing plate 5 and the exchange tube 4 to determine the first pressure data. By comparing the pressure data of the closing plate 5, it is determined whether a blockage occurs inside the first tube 21 to remind the operator to adjust the surgical process, so as to facilitate operations when the first tube 21 maintains normal negative pressure suction, thereby reducing cases where fluids, residual blood, smoke, etc., in the abdominal cavity cannot be sucked out in a timely manner, and maintaining the surgical field of view.

The control panel is further electrically connected to a second pressure sensor, and the second pressure sensor is located between the two sides of the sleeve 41 and the first spring 42; the second pressure sensor is configured to measure second pressure data when the sleeve 41 applies pressure in real time and send the second pressure data to the control panel; the control panel is configured to compare the second pressure data with a set second standard value, if the second pressure data is greater than the second standard value, a warning instruction is sent to the speaker 11, and if the second pressure data is less than the second standard value, a standby instruction is sent to the speaker 11.

For example, during the movement of the sleeve 41, the condition of the second pressure data when pressure is applied to both sides of the sleeve 41 is detected to determine whether the sleeve 41 moves, so as to remind the operator that the negative pressure suction force inside the first tube 21 is too high, and remind the operator to perform active adjustment, reducing the occurrence of continuous increase of the negative pressure suction force during the continuous operation of the operator, thereby ensuring that the interior of the first tube 21 is in a normal negative pressure suction force state.

### Embodiment 5

The difference from Embodiment 4 is that a first electromagnetic valve 34 communicates with the suction port 33, the first electromagnetic valve 34 is electrically connected to the control panel, and a normal state of the first electromagnetic valve 34 is a normally open state; a cleaning pipe 23 communicates between an end of the first tube 21 close to the suction port 33 and the second tube 22, a second electromagnetic valve 24 communicates within the cleaning pipe 23, the second electromagnetic valve 24 is electrically connected to the control panel, and a normal state of the second electromagnetic valve 24 is a normally closed state.

It further includes a locking mechanism, the locking mechanism includes a locking shaft 7 corresponding to the closing plate 5, and the locking shaft 7 is rotatably matched with the suction tube 2; a moving cavity (not shown in the figure) is opened within the suction tube 2, the moving cavity is located at one side of the shaft 51, a first magnet layer (not shown in the figure) is fixedly connected to an end of the locking shaft 7 away from the shaft 51, the first magnet layer is located within the moving cavity, and an electromagnet (not shown in the figure) is disposed within the moving cavity, the electromagnet being electrically connected to the control panel; when the electromagnet is not energized, the locking shaft 7 is separated from the closing plate 5; when the electromagnet is energized, the locking shaft 7 abuts against the closing plate 5.

The specific implementation process is as follows: When the interior of the first tube 21 needs to be cleaned, the locking shaft 7 is first used to abut against the closing plate 5 to fix the position of the closing plate 5 so that it does not change.

The opening and closing of the first electromagnetic valve 34 and the second electromagnetic valve 24 are controlled through the control panel to realize the closing of the first tube 21 and the communication between the first tube 21 and the second tube 22, so as to use the irrigation fluid of the second tube 22 to wash the impurities remaining inside the first tube 21, thereby ensuring the patency of the first tube 21 and reducing the residue of impurities attached inside the first tube 21.

Obviously, the above embodiments are only examples for clear description and are not limitations on the implementations. For those of ordinary skill in the art, other changes or modifications in different forms can be made based on the above description. It is unnecessary and impossible to list all implementations here. The obvious changes or modifications derived therefrom still fall within the protection scope of the present invention.

1. A multi-functional laparoscopic aspirator, comprising a holding part (1) and a suction tube (2), wherein the holding part (1) is fixedly connected to the suction tube (2), the suction tube (2) communicates with a negative pressure pump for generating negative pressure suction through a pipe (13), and the suction tube (2) also communicates with a compression pump for pumping irrigation fluid through a pipe (13), wherein the suction tube (2) includes a first tube (21) and a second tube (22), the first tube (21) communicates with the negative pressure pump through a pipe (13), and the second tube (22) communicates with the compression pump through a pipe (13);
a clamping mechanism for clamping surrounding tissues is disposed at an end of the suction tube (2) away from the holding part (1);
the first tube (21) is located above the second tube (22), and a pressure conversion mechanism for communicating based on a pressure difference between an interior of the first tube (21) and an interior of the second tube (22) is communicated between the first tube (21) and the second tube (22); and a suction port (33) communicates with an end of the first tube (21) away from the negative pressure pump, and a number of nozzles (32) communicate with an end of the second tube (22) away from the compression pump.

2. The multi-functional laparoscopic aspirator according to claim 1, wherein the clamping mechanism includes a first block (3) and a second block (31), the second block (31) is located above the first block (3), the first block (3) is fixedly connected to the suction tube (2), and the second block (31) is rotatably matched with the suction tube (2);
the nozzles (32) are located at a side of the second block (31) close to the first block (3), and the suction port (33) is located at an end of the second block (31) away from the suction tube (2);
a press handle (12) is rotatably matched with an end of the holding part (1) away from the suction tube (2), an extension block fixedly connected to the holding part (1) is further disposed at one side of the press handle (12), a balloon (15) is disposed between the extension block and the press handle (12), and the balloon (15) communicates with a gas pipe (16); an end of the gas pipe (16) away from the balloon (15) communicates with a pneumatic telescopic rod (17), the pneumatic telescopic rod (17) is hinged to an end of the second block (31) away from the suction port (33), and a first torsion spring for resetting is disposed at a rotation position of the second block (31) and the suction tube (2); and
when the press handle (12) squeezes the balloon (15), the pneumatic telescopic rod (17) extends out and the second block (31) moves away from the first block (3); when the balloon (15) resets to push the press handle (12) to move, the pneumatic telescopic rod (17) retracts and the second block (31) moves close to the first block (3).

3. The multi-functional laparoscopic aspirator according to claim 2, wherein the pressure conversion mechanism includes a number of exchange tubes (4) and a closing plate (5), and the exchange tubes (4) are located between the first tube (21) and the second tube (22); a locking slot is disposed at a bottom of each exchange tube (4), a diameter of the locking slot is greater than a diameter of the exchange tube (4), the closing plate (5) is rotatably matched with the suction tube (2), and the closing plate (5) is located within the locking slot;
a shaft (51) is fixedly connected to one side of the closing plate (5), the shaft (51) is rotatably matched with the suction tube (2), a second torsion spring is sleeved on the shaft (51), a sleeve (41) is slidably matched within the exchange tube (4), and a first spring (42) is fixedly connected between two sides of the sleeve (41) and the first tube (21); and
when a negative pressure suction force within the first tube (21) is in a normal state, a top of the sleeve (41) is located within the exchange tube (4); when the negative pressure suction force within the first tube (21) is greater than the normal state, the top of the sleeve (41) is located within the first tube (21).

4. The multi-functional laparoscopic aspirator according to claim 3, wherein the shaft (51) of the closing plate (5) is located at a side of the second tube (22) close to the compression pump.

5. The multi-functional laparoscopic aspirator according to claim 4, wherein a shaking plate (25) corresponding to the exchange tube (4) is disposed at a top of the first tube (21), a loosening slot is opened at the top of the first tube (21), the shaking plate (25) is slidably matched with the loosening slot, and a number of second springs (26) are connected between the shaking plate (25) and the suction tube (2).

6. The multi-functional laparoscopic aspirator according to claim 5, wherein an auxiliary cavity is further opened within the suction tube (2), the auxiliary cavity is located between adjacent exchange tubes (4), and the auxiliary cavity is located above the closing plate (5) close to the shaft (51), a pushing block (52) is fixedly connected to the shaft (51), and the pushing block (52) is slidably matched with the auxiliary cavity; a pushing rod (6) is slidably matched within the auxiliary cavity, the pushing rod (6) is located above the shaft (51), and a third spring (61) is sleeved on the pushing rod (6); and
when the closing plate (5) is perpendicular to the exchange tube (4), the pushing block (52) is separated from the pushing rod (6); when the closing plate (5) is parallel to the exchange tube (4), the pushing block (52) abuts against the pushing rod (6).

7. The multi-functional laparoscopic aspirator according to claim 6, wherein it further includes a control panel located inside the holding part (1), the control panel is electrically connected to a first pressure sensor and a speaker (11), the first pressure sensor is located at a side of the closing plate (5) close to the exchange tube (4), and the speaker (11) is located at one side of the holding part (1); and
the first pressure sensor is configured to measure first pressure data at a top of the closing plate (5) in real time and send the first pressure data to the control panel, the control panel is configured to compare the first pressure data with a set first standard value, if the first pressure data is greater than the first standard value, a standby instruction is issued to the speaker (11), and if the first pressure data is less than the first standard value, a reminder instruction is sent to the speaker (11).

8. The multi-functional laparoscopic aspirator according to claim 7, wherein the control panel is further electrically connected to a second pressure sensor, and the second pressure sensor is located between the two sides of the sleeve (41) and the first spring (42); and
the second pressure sensor is configured to measure second pressure data when the sleeve (41) applies pressure in real time and send the second pressure data to the control panel; the control panel is configured to compare the second pressure data with a set second standard value, if the second pressure data is greater than the second standard value, a warning instruction is sent to the speaker (11), and if the second pressure data is less than the second standard value, a standby instruction is sent to the speaker (11).

9. The multi-functional laparoscopic aspirator according to claim 8, wherein a number of buttons (14) for adjusting working power of the negative pressure pump and the compression pump are disposed on the press handle (12), and the buttons (14) are located at upper and lower sides of the press handle (12).

10. The multi-functional laparoscopic aspirator according to claim 9, wherein the nozzles (32) are located at two sides of a top of the second block (31).

11. The multi-functional laparoscopic aspirator according to claim 10, wherein a first electromagnetic valve (34) communicates with the suction port (33), the first electromagnetic valve (34) is electrically connected to the control panel, and a normal state of the first electromagnetic valve (34) is a normally open state; and
a cleaning pipe (23) communicates between an end of the first tube (21) close to the suction port (33) and the second tube (22), a second electromagnetic valve (24) communicates within the cleaning pipe (23), the second electromagnetic valve (24) is electrically connected to the control panel, and a normal state of the second electromagnetic valve (24) is a normally closed state.

12. The multi-functional laparoscopic aspirator according to claim 11, wherein it further includes a locking mechanism, the locking mechanism includes a locking shaft (7) corresponding to the closing plate (5), and the locking shaft (7) is rotatably matched with the suction tube (2);
a moving cavity is opened within the suction tube (2), the moving cavity is located at one side of the shaft (51), a first magnet layer is fixedly connected to an end of the locking shaft (7) away from the shaft (51), the first magnet layer is located within the moving cavity, and an electromagnet is disposed within the moving cavity, the electromagnet being electrically connected to the control panel; and
when the electromagnet is not energized, the locking shaft (7) is separated from the closing plate (5); when the electromagnet is energized, the locking shaft (7) abuts against the closing plate (5).

13. The multi-functional laparoscopic aspirator according to claim 12, wherein a number of anti-slip grooves are opened on the extension block.
